(19) United States
(12) Patent Application Publication
(10) Pub. No.: US 2026/0365916 A1
(43) Pub. Date: Nov. 19, 2026

(54) BACTERIAL SPORES AS GREEN AND EFFICIENT BIOLOGICAL-INORGANIC HYBRID PHOSPHORS

(71) Applicant: Temasek Life Sciences Laboratory Limited, Singapore (SG)

(72) Inventors: Shen-Yi Ian Cheong; Ajitha Sundaresan

(21) Appl. No.: 19/142,855

(22) PCT Filed: Dec. 26, 2023
(86) PCT No.: PCT/SG2023/050861

§ 371 Date: Jun. 24, 2025

(30) Foreign Application Priority Data
Dec. 27, 2022 (SG) 10202260603W
Oct. 24, 2023 (SG) 10202303014R

(51) Int. Cl. C09K 11/02; C09K 11/06; H10H 20/851

(52) U.S. Cl. CPC C09K 11/02; C09K 11/06; H10H 20/8512; C09K 2211/181; C09K 2211/182

(57) ABSTRACT

Rare earth elements (REEs) are raw materials of high importance for modern technologies such as light emitting diodes (LEDs), solar panels, and photovoltaic cells. These materials are associated with geopolitical supply risks, environmental concerns and recycling challenges. Increasing the efficiency of their performance, simplifying the manufacture, and recycling of lanthanide-based phosphors are key pillars for addressing these challenges. This study introduces Bacillus and Clostridium bacterial spores as a biological material with the remarkable ability to sequester lanthanides and function as phosphors. The addition of an antenna molecule such as thenoyltrifluoroacetone (TTA) increases the quantum yield of the phosphor, while also specifically increasing the amount of quantity of lanthanide sequestered by Clostridium spores. Adding strontium as a co-dopant to the spore further doubles the luminescent output from the spores. Different colours are achieved by varying the lanthanide composition. Ultraviolet LEDs coated with spore phosphors have far higher conversion efficiencies than phosphor controls. Besides superior performance, the inert nature of spores facilitates easy manufacturing and recycling, making spores a potential ally in our quest to minimize environmental impact by maximizing the use of precious and contested lanthanide resources.

### FIELD OF INVENTION

The current invention relates to a phosphor material and a method of forming the phosphor material, a composite material comprising the phosphor material, and a modified light emitting diode (LED) comprising the composite material.

### BACKGROUND

The listing or discussion of a prior-published document in this specification should not necessarily be taken as an acknowledgement that the document is part of the state of the art or is common general knowledge.

Phosphors are substances which are luminescent in a very specific way, called phosphorescence, which means that they can emit light for a long duration (ranging from microseconds to even milliseconds or longer) after being excited by energy. Phosphors generally consist of a host material or compound that can absorb energy and transfer it to an activator, which is the substance which actually emits light. When the activator receives energy from the host, it becomes excited and moves to a higher energy singlet or triplet state. As the activator ion returns to its lower energy state, it releases the excess energy in the form of photons, which is what we perceive as light. The role of the host is to provide an environment that can efficiently transfer the absorbed energy to the activator ion. The exact properties of the emitted phosphorescent light depends on energy transitions between the host material and activator material.

Phosphors are constructed by doping the host material with the activator material. Most commercially available phosphors are doped with transition metals or rare earth ions as activators. Compared to transition metals, rare earth ions (also known as lanthanide ions) contain a large number of unpaired electrons in their 4f orbitals, shielding their valence 6 s electrons. This shielding effect minimizes vibrational losses to the environment, resulting in sharp line-like peaks in the emission spectra. Further, emissions that occur due to electronic transitions in these 4f-f orbitals are parity forbidden, giving rise to prolonged phosphorescence lifetimes. Another important property of lanthanide ions is their unusually long Stokes shifts which improves emission efficiency by reducing self-absorption/quenching effects. Due to such favourable luminescence properties, lanthanide-based phosphors have transformed just about every application involving photonic output (e.g. lighting, display technology, biological assays, and lasers) but also photonic input (e.g. medical imaging, photovoltaic cells).

However, several challenges can affect the market for these materials. These challenges include the following.
1. Resource Scarcity and Geopolitical Risks: The supply of a significant portion of the world's rare-earth elements originates from very few countries only, thus making most other countries being heavily reliant on their geopolitical stability for a steady supply.
2. Environmental Concerns: The extraction and processing of rare-earth elements often involve environmentally harmful processes, including the release of radioactive waste. This has led to increased scrutiny from environmental regulators, and the costs associated with meeting stricter environmental standards could impact the market.
3. Recycling Challenges: Rare earth elements are difficult and expensive to recycle, which can contribute to supply issues. More efficient recycling processes could help alleviate this issue but developing such technologies could take time and require significant investment.
4. Colour Quality: While LED technology has improved significantly, reproducing the warm light quality that many consumers prefer from incandescent bulbs is still a challenge. Advances in phosphor technology are helping to address this issue, but it remains as a concern.

5. Supply Chain Issues: Many of the materials used in LED production, such as rare-earth elements, are subject to price volatility and supply chain disruptions. This is particularly true in times of geopolitical tension or global economic uncertainty.

To efficiently function with a high quantum yield for the varied applications, rare earth based phosphors require an appropriate host material which can maximize energy absorption. A good host material should satisfy several properties (Zhuo, Y. et al., Nat. Commun. 2018, 9, 4377; Hermus, M. & Brgoch, J. The Electrochemical Society Interface, 2015, 24, 55). Besides being compatible with dopant ions, they should have low phonon energy, high transparency, high chemical stability, high mechanical strength, preferably high refractive index, and a suitable energy gap to match the energy levels of the dopant ions and facilitate efficient energy transfer. With such a long list of critical parameters, it is not surprising that the most common host materials have been inorganic in nature, like garnets, perovskites, nitrides, silicates and borates. Inorganic lattices allow tunability of phosphorescence properties by modification of the lattice composition (Zhao, M. et al., Sci. Adv. 2019, 5, eaav0363; Wang, S.-S. et al., J. Am. Chem. Soc., 2013, 135, 12504) and rare earth dopant concentrations (Malik, C. et al., Radiat. Phys. Chem., 2020, 168, 108561; Zhang, J. et al., Inorg. Chem., 2020, 59, 2241). However, onerous and complex manufacturing conditions are required to achieve the right lattice composition, such as high temperature (Lavat, A. et al., Cryst. Res. Technol., 2004, 39, 840; Upadhyay, K. et al., Superlattices Microstruct., 2015, 78, 116; Chen, X. et al., Minerals, 2017, 7, 44), pressure (Panda, D. P. et al., J. Mater. Chem., 2022, 10, 16723; Atuchin, V. V. et al., ACS Appl. Mater. Interfaces, 2015, 7, 26235) or caustic agents like ammonium hydroxide and nitric acid (Gao, W. et al., J. Rare Earths, 2009, 27, 886). In some cases, their production also generates tremendous quantities of heat or toxic gases which negatively impact the environment (Deyneko, D. V. et al., J. Alloys Compd., 2021, 887, 161340). Other disadvantages of inorganic hosts include limited tunability, difficulty of integration into flexible or thin-film formats, reabsorption of emission from dopant ions, and finally, in the case of nanoscale inorganic phosphors, size and shape control (Chiriu, D. et al., Phys. Status Solidi C, 2016, 13, 989).

These problems have led to the development of organic materials with improved tunability, flexibility and photonic characteristics which cover some of the deficiencies of inorganic materials (Zhao. Y.-W. et al., ACS Appl. Mater. Interfaces, 2016, 8, 24123; Song, B. et al., ACS Appl. Mater. Interfaces, 2020, 12, 6137; Lu, Y. & Yan, B., Chem. Commun., 2014, 50, 15443; Zhang, N.-N. et al., Chem. Commun., 2017, 53, 9269). However, organic materials come with their own hosts of problems. Not surprisingly, organic materials have generally lower thermal and chemical stability than their inorganic counterparts. They are often sensitive to oxygen and moisture, which can quench phosphorescence and degrade the material. Thus devices are required to be encapsulated to prevent such exposure to air. Non-radiative energy dissipation due to vibrations and oscillations also makes their intersystem crossing transitions quite fast, thus shortening the phosphorescence lifetimes of many organic phosphors.

Bacterial spores are dormant, hardy structures built by nature to endure harsh environmental conditions.

Prior work on bacteria and lanthanides were focused only on the ability of bacteria to sequester lanthanides for bioremediation. Specifically, there have only been a few documents to our knowledge which mentioned the accumulation of lanthanides in spores in the context mining/extracting or bioremediation. (Dong, W. et al. Minerals, 2022, 12, 866; Dong, W. et al., Appl. Environ. Microbiol., 2019, 85, e00956-19; Cockell, C. S. et al., Nat. Commun., 2020, 11, 5523).

We are not aware of prior art that combines spores with lanthanides to achieve luminescence. There are many studies which used bacterial spores to recycle lanthanides, but none of them have actually shown phosphor functionality.

### SUMMARY OF INVENTION

It has been surprisingly found that similar results to current phosphor technologies can be obtained by a lower quantity of phosphors through their incorporation into bacterial spores. This can be achieved by improving the efficiency of the rare-earth phosphorescence, which will maximize the use of this scare resource and may help to alleviate the issues associated with the environmental and recycling challenges associated with these rare-earth metals. A further advantage is that the manufacturing processes needed to make such phosphors are easy to conduct, less energy-intensive and are better for the environment.

Aspects and embodiments of the invention will now be described by reference to the following numbered clauses.
1. A phosphor material comprising:
   a bacterial spore as a host material; and
   a lanthanide dopant within the host material and/or on a surface of the host material, wherein:
      the bacterial spore is in a dormant state; and
      the bacterial spore is selected from one or more bacterial spores selected from one or more of the Bacillus and Clostridium genera.
2. The phosphor material according to Clause 1, wherein the lanthanide dopant is selected from one or more of the group consisting of Eu³⁺, Sm³⁺, Tb³⁺, and Dy³⁺, optionally wherein the lanthanide dopant is selected from one or more of the group consisting of Eu³⁺, Sm³⁺, and Tb³⁺ (e.g. Eu³⁺).
3. The phosphor material according to Clause 1 or Clause 2, wherein the bacterial spores are formed from one or more of Bacillus megaterium, Bacillus cereus, Bacillus subtilis, Clostridium acetobutylicum, Clostridium butyricum, Clostridium botulinium, Clostridium difficile, Clostridium novyi-NT and Clostridium septicum CS11, optionally wherein the bacterial spores are formed from one or more of Bacillus megaterium, Clostridium novyi-NT and Clostridium septicum (e.g. one or both of Clostridium novyi -NT and Clostridium septicum).
4. The phosphor material according to any one of the preceding clauses, wherein the phosphor material further comprises an antenna molecule that is complexed to the lanthanide dopant.
5. The phosphor material according to Clause 4, wherein the antenna molecule is selected from one or more of the group consisting of a beta-diketone and triphenylphosphine oxide.
6. The phosphor material according to Clause 4, wherein the antenna molecule an aromatic beta-diketone, optionally wherein the aromatic beta-diketone is selected from one or more of the group consisting of thenoyltrifluoroacetone (TTA), 1-(-2-Naphthoyl)-3,3,3-trifluoroacetone (NTFA), and 4,4,4-Trifluoro-1-phenyl-1,3-butanedione (BTFA).
7. The phosphor material according to any one of Clauses 4 to 6, wherein the antenna molecule is complexed to the lanthanide dopant, said complex having the formula Ln[ANT]ₙ, where Ln is a member of the lanthanide series of elements, ANT is an antenna molecule and n is a whole number integer, optionally wherein n is from 3 to 300, such as 30.
8. The phosphor material according to Clause 7, wherein Ln[ANT]ₙ is selected from one or more of the group consisting of Eu[TTA]₃, Sm[TTA]₃, Dy[TTA]₃, Tb[TTA]₃, Eu[BTFA]₃, Eu[NTFA]₃, optionally wherein Ln[ANT]ₙ is selected from one or more of the group consisting of Eu[TTA]₃, Sm[TTA]₃, and Tb[TTA]₃ (e.g. Eu[TTA]₃, and Tb[TTA]₃, such as and Eu[TTA]₃).
9. The phosphor material according to any one of Clauses 4 to 8, wherein the molar ratio of the lanthanide dopant to the antenna molecule is from 1:300 to 1:3, such as from 1:200 to 1:10, such as from 1:100 to 1:20, such as about 1:30.
10. The phosphor material according to any one of the preceding clauses, wherein the phosphor material further comprises a co-dopant material, optionally wherein the co-dopant material is selected from one or more of the group consisting of Sr²⁺ and Ca²⁺.
11. The phosphor material according to any one of the preceding clauses, wherein the lanthanide dopant is present in an amount of from 20 to 200 nmol per mg dry spore biomass.
12. A composite material comprising:
   a phosphor material as described in any one of Clauses 1 to 11; and
   a bulk material, wherein the bulk material is selected from one or more of the group consisting of a resin, a coating, an adhesive, and an encapsulating material and the bulk material is optically transparent or optically translucent to the excitation and emission wavelengths of the phosphor material.
13. The composite material according to Clause 12, wherein the bulk material comprises an adhesive, where the adhesive is one that is optically transparent to the excitation and emission wavelengths of the phosphor material, optionally wherein the adhesive is Norland™ 88.
14. A modified light emitting diode, comprising:
   a light emitting diode, having a light emitting diode bulb section; and
   a cured composite material comprising a phosphor material as described in any one of Clauses 1 to 11 and an optically clear adhesive covering a surface of the light emitting diode section, such that light from the light emitting diode passes through the cured composite material.
15. The modified light emitting diode according to Clause 14, wherein the light emitting diode is an ultraviolet light emitting diode.
16. A method of forming a phosphor material as described in any one of Clauses 1 to 11, the method comprising the steps of:
(a) providing a plurality of bacterial spores of the Bacillus and Clostridium genera; and
(b) adding a lanthanide material to the plurality of bacterial spores to provide the phosphor material, wherein the lanthanide material is either a lanthanide salt or a lanthanide complexed to an antenna molecule.
17. The method according to Clause 16, wherein the lanthanide material is provided in a solvent to the bacterial spores.
18. The method according to Clause 17, wherein the solvent comprises water and ethanol, optionally wherein ethanol forms from 1 to 50% vol/vol, such as about 3% vol/vol.
19. The method according to Clause 17 or Clause 18, wherein the lanthanide material is provided in the solvent at a concentration of from 0.005 mM to 10 mM, such as from 0.01 mM to 3 mM, such as from 0.015 mM to 1.5 mM, such as from 0.15 mM to 0.75 mM.
20. The method according to any one of Clauses 17 to 19, wherein the antenna molecule, when present, is provided in an amount of from 0.1 mM to 10 mM, such as about 4.5 mM.
21. The method according to any one of Clauses 17 to 20, wherein the method further comprises the step of substantially removing the solvent from the phosphor material.

### DRAWINGS

FIG. 1 Depicts the General Conceptual Idea of This Invention.

FIG. 2 depicts that bacterial spores sequester lanthanides in the presence of beta-diketones. 0.14 mM Europium (Eu) with 4.2 mM TTA, or Eu 0.14 mM alone were added to dormant, germinated and overnight vegetative cultures of each bacteria, and the Eu content of the supernatants were measured using inductively coupled plasma optical emission spectroscopy (ICP-OES). Graphs represent Eu content per biomass weight (nmol/mg) for different spore and vegetative forms of B. subtilis (A), B. thuringiensis (B), C. novyi-NT (C), C. septicum -CS11 (D) both in the presence (circle) and absence (square) of TTA. Data represents individual values with Mean±SD, n=2 biological replicates.

FIG. 3 depicts that Clostridium spore phosphors exhibit high phosphorescence in the presence of the beta-diketone TTA. Spore phosphors were prepared by drying Eu or Eu-TTA loaded spores in a UV transparent 384-well microtiter plate under high vacuum and their fluorescence was measured. (A) Image of dried B. subtilis (Column 1), B. thuringiensis (Column 3), C. novyi-NT (Column 5), C. septicum CS11 (Column 7) spore phosphors and no spore phosphor control (Column 9) doped with 1.4 mM (Rows 1,2) and 0.14 mM (Rows 3,4) Eu or Eu-TTA (4.2 mM) excited at 365 nm using a ultraviolet (UV) transilluminator. Solvent refers to 3% ethanol. Image obtained using a Samsung S10 plus smartphone. (B) Fluorescence intensity values of spore phosphors at 620 nm for all bacteria at different concentrations of Eu (1.4 mM (triangle), 0.14 mM (circle), 0.014 mM (square)) in the presence and absence of TTA (4.2 mM) plotted against the fluorescence lag times. All samples were excited at 370 nm. Data represents Mean±SEM, n=4 biological replicates.

FIG. 4 depicts that among Bacillus spore phosphors, B. megaterium exhibits the highest phosphorescence in the presence of the beta-diketone TTA. Spore phosphors were prepared by drying Eu or Eu-TTA loaded spores in a UV transparent 384-well microtiter plate under high vacuum and their fluorescence was measured. (A) Image of dried B. subtilis (Column 1), B. thuringiensis (Column 4), B. cereus (Column 7), B. megaterium (Column 10), C. novyi-NT (Column 13), C. septicum CS11 (Column 16) spore phosphors and no spore phosphor control (Column 19) doped with 0.14 mM Eu or Eu-TTA (4.2 mM) excited at 365 nm using a UV transilluminator. Solvent refers to 3% ethanol. Image obtained using a Google pixel 6a smartphone. (B) Fluorescence intensity values of Eu 0.14 mM spore phosphors at 620 nm for all bacteria in the presence (circle) and absence (triangle) of TTA (4.2 mM) plotted against the fluorescence lag times. All samples were excited at 370 nm. Data represents Mean±SEM, n =2 biological replicates.

FIG. 5 depicts the excitation-emission matrix (EEM) for europium spore phosphors. Spore phosphors were prepared by either drying Eu or Eu-TTA loaded spores in a UV transparent 384-well microtiter plate under high vacuum or by using their solution form prior to drying and their EEM was measured. (A) EEM contour plot for solid (left) and liquid (right) C. novyi-NT, C. septicum CS11 and no spore control phosphors loaded with Eu (1.4 mM)-TTA (4.2 mM). (B) EEM contour plot for solid (left) and liquid (right) C. novyi-NT, C. septicum CS11 and no spore control phosphors loaded with Eu (1.4 mM). Intensity scale refers to fluorescence intensity on a log₁₀ scale.

FIG. 6 depicts that Clostridium spore phosphors have long shelf life in the order of months. Spore phosphors were prepared by drying Eu or Eu-TTA loaded spores in a UV transparent 384-well microtiter plate under vacuum and their fluorescence was monitored at specific timepoints. Fluorescence intensity values of spore phosphors at 620 nm, lag time 20 us for all bacteria at different concentrations of Eu (1.4 mM, 0.14 mM) in the presence and absence of TTA (4.2 mM) plotted against the number of weeks at which the measurement was done. All samples were excited at 370 nm. Data represents Mean±SEM, n =4 biological replicates.

FIG. 7 depicts that doping different lanthanides produces differently coloured spore phosphors. Spore phosphors were prepared by drying Europium (Eu)/Samarium (Sm)/Terbium (Tb)/Dysprosium (Dy) or Eu-TTA/Sm-TTA/Tb-TTA/Dy-TTA loaded spores in polymerase chain reaction (PCR) tubes. (A) Image of dried C. novyi-NT spore phosphors doped with 0.14 mM of respective Ln or Ln-TTA (4.2 mM) excited at 254 nm (top) and 365 nm (bottom) using a UV transilluminator. (B) Image of dried C. septicum-CS11 spore phosphors doped with 0.14 mM of respective Ln or Ln-TTA (4.2 mM) excited at 254 nm (top) and 365 nm (bottom) using a UV transilluminator. Images were obtained using the Google Pixel 6a smartphone.

FIG. 8 depicts the excitation-emission matrix (EEM) for samarium spore phosphors. Spore phosphors were prepared by either drying Sm or Sm-TTA loaded spores in a UV transparent 384-well microtiter plate under high vacuum or by using their solution form prior to drying and their EEM was measured. (A) EEM contour plot for solid (left) and liquid (right) C. novyi-NT, C. septicum CS11 and no spore control phosphors loaded with Sm (1.4 mM)-TTA (4.2 mM). (B) EEM contour plot for solid (left) and liquid (right) C. novyi-NT, C. septicum CS11 and no spore control phosphors loaded with Sm (1.4 mM). Intensity scale refers to fluorescence intensity on a log₁₀ scale.

FIG. 9 depicts the excitation-emission matrix (EEM) for terbium spore phosphors. Spore phosphors were prepared by either drying Tb or Tb-TTA loaded spores in a UV transparent 384-well microtiter plate under high vacuum or by using their solution form prior to drying and their EEM was measured. (A) EEM contour plot for solid (left) and liquid (right) C. novyi-NT, C. septicum CS11 and no spore control phosphors loaded with Tb (1.4 mM)-TTA (4.2 mM). (B) EEM contour plot for solid (left) and liquid (right) C. novyi-NT, C. septicum CS11 and no spore control phosphors loaded with Tb (1.4 mM). Intensity scale refers to fluorescence intensity on a log₁₀ scale.

FIG. 10 depicts that co-doping of strontium with Eu-TTA spore phosphors increases their fluorescence. Spore phosphors were prepared by drying Eu-Sr-TTA or Eu-TTA loaded spores in PCR tubes or a UV transparent 384-well microtiter plate under high vacuum and their fluorescence was measured. (A) Image of dried C. novyi-NT spore phosphors doped with 0.06 mM and 1.9 mM Eu-Sr (476 mM)-TTA (5.4 mM) or 0.06 mM and 1.9 mM Eu-TTA (5.4 mM) in PCR tubes excited at 365 nm using a UV transilluminator with (Top) and without (Bottom) a longpass filter. Images were obtained using a Samsung S10 plus smartphone. Fluorescence intensity values of C. novyi-NT spore phosphors at 620 nm at concentrations of Eu 1.9 mM (B) and Eu 0.06 mM (C) with TTA (5.4 mM) in the presence (circle) and absence (triangle) of strontium (476 mM) plotted against the fluorescence lag times. All samples were excited at 370 nm. Mean±SD, n=6 biological replicates.

FIG. 11 depicts the comparison of Eu-TTA spore phosphor with commercial yttrium oxide red phosphor. Solid spore phosphors and yttrium oxide phosphors were prepared by drying Eu (0.14 mM)-TTA (4.2 mM) loaded spores and yttrium oxide suspensions (containing 0.14 mM Eu) in a UV transparent 384-well microtiter plate under high vacuum respectively and their fluorescence was measured. (A) Image of solid C. novyi-NT spore phosphors (Column 2 solid, Column 6 liquid) doped with 0.14 mM Eu-TTA (4.2 mM) and yttrium oxide phosphor containing 0.14 mM Eu (Column 4 solid, Column 8 liquid) excited at 254 nm using a UV transilluminator. Image was obtained using a Google Pixel 6 Pro smartphone. (B) Fluorescence intensity values of spore phosphors and yttrium oxide phosphor in the liquid and solid state at 250 nm excitation. (C) Fluorescence intensity values of spore phosphors and yttrium oxide phosphor in the liquid and solid state at 370 nm excitation. Fluorescence intensity values for B and C measured at 620 nm, no lag time. Data represents individual values with Mean±SD, n=3 biological replicates.

FIG. 12 depicts that coating of UV LEDs with Ln-TTA spore phosphors produces different coloured LEDs. Spore phosphors were prepared by drying Eu (0.14 mM)-TTA (4.2 mM) or Tb (0.14 mM)-TTA (4.2 mM) loaded spores under vacuum and were mixed with UV transparent adhesive Norland 88 for coating on to UV LEDs. (A) Images of dried Eu-TTA coating and C. novyi-NT spore phosphor doped with 0.14 mM Eu-TTA (4.2 mM) coating on UV LEDs excited at 365 nm using a 9V battery. (B) Images of dried Eu or Eu-TTA coating and C. novyi-NT spore phosphor doped with 0.14 mM Eu-TTA (4.2 mM) or 0.14 mM Eu coating on UV LEDs excited at 365 nm using a 9V battery. Solvent refers to 3% ethanol. (C) Images of dried Tb or Tb-TTA coating and C. novyi-NT spore phosphor doped with 0.14 mM Tb-TTA (4.2 mM) or 0.14 mM Tb coating on UV LEDs excited at 275 nm using 9V batteries in series. Solvent refers to 50% ethanol. Images obtained using a Google pixel 6 Pro smartphone (A) and a Nikon COOLPIX L120 DSLR camera with and without long pass filter for (B) and (C) respectively.

### DESCRIPTION

Of the subject matter encompassed by the present disclosure. Accordingly, the subject matter of this disclosure is not intended to be limited to the forms or embodiments so described.

It has been surprisingly found that bacterial spores to play the role of host materials for rare earth metals, thereby providing surprisingly good phosphors. Bacillus and Clostridium spores have been found to efficiently sequester lanthanides and act as efficient phosphors. We have surprisingly found that preparation of the phosphors is trivial and involves the mere addition of lanthanides to spores in solution. Phosphorescence may be further enhanced by adding an antenna molecule and a co-dopant. Based on the current results, it appears that Clostridium strains may outperform Bacillus strains, both in terms of phosphorescent output, and shelf-life. A basic application is demonstrated by coating ultraviolet (UV) LEDs with spore phosphors to achieve efficient conversion of UV light to red light.

Thus, in a first aspect of the invention, there is provided a phosphor material comprising:
a bacterial spore as a host material; and
a lanthanide dopant within the host material and/or on a surface of the host material, wherein:
   the bacterial spore is in a dormant state; and
   the bacterial spore is selected from one or more bacterial spores selected from one or more of the Bacillus and Clostridium genera.

In embodiments herein, the word “comprising” may be interpreted as requiring the features mentioned, but not limiting the presence of other features. Alternatively, the word “comprising” may also relate to the situation where only the components/features listed are intended to be present (e.g. the word “comprising” may be replaced by the phrases “consists of” or “consists essentially of”). It is explicitly contemplated that both the broader and narrower interpretations can be applied to all aspects and embodiments of the present invention. In other words, the word “comprising” and synonyms thereof may be replaced by the phrase “consisting of” or the phrase “consists essentially of” or synonyms thereof and vice versa.

The phrase, “consists essentially of” and its pseudonyms may be interpreted herein to refer to a material where minor impurities may be present. For example, the material may be greater than or equal to 90% pure, such as greater than 95% pure, such as greater than 97% pure, such as greater than 99% pure, such as greater than 99.9% pure, such as greater than 99.99% pure, such as greater than 99.999% pure, such as 100% pure.

As used herein, the singular forms “a,” “an,” and “the” include plural referents unless the context clearly dictates otherwise. Thus, for example, reference to “a composition” includes mixtures of two or more such compositions, and the like.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 0.01 to 2.0” should be interpreted to include not only the explicitly recited values of about 0.01 to about 2.0, but also include individual values and sub-ranges within the indicated range. Thus, included in this numerical range are individual values such as 0.5, 0.7, and 1.5, and sub-ranges such as from 0.5 to 1.7, 0.7 to 1.5, and from 1.0 to 1.5, etc. Furthermore, such an interpretation should apply regardless of the breadth of the range or the characteristics being described. Additionally, it is noted that all percentages are in weight, unless specified otherwise.

As used herein, a plurality of compounds, elements, or steps may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

As noted hereinbefore, it has been found that bacterial spores may be used as a suitable host material to enhance the phosphorescent properties of lanthanide dopant materials. Said lanthanide dopant materials may be within the bacterial spore, on the surface of the bacterial spore or both. In the current invention, the bacterial spore is provided in a dormant state, as this is more conducive to inclusion in displays and consumer electronics. Any suitable bacterial spore may be used herein. For example, the bacterial spore may be selected from one or more bacterial spores selected from one or more of the Bacillus and Clostridium genera.

The lanthanide dopant material may be any suitable material. For example, the lanthanide dopant may be selected from one or more of the group consisting of Eu³⁺, Sm³⁺, Tb³⁺, and Dy³⁺. In particular embodiments of the invention, the lanthanide dopant may be selected from one or more of the group consisting of Eu³⁺, Sm³⁺, and Tb³⁺ (e.g. Eu³⁺).

The bacterial spores may be formed from one or more of Bacillus megaterium, Bacillus cereus, Bacillus subtilis, Clostridium acetobutylicum, Clostridium butyricum, Clostridium botulinium, Clostridium difficile, Clostridium novyi-NT and Clostridium septicum CS11, optionally wherein the bacterial spores are formed from one or more of Bacillus megaterium, Clostridium novyi-NT and Clostridium septicum (e.g. one or both of Clostridium novyi-NT and Clostridium septicum).

In particular embodiments of the invention that may be mentioned herein, the bacterial spores may be selected from the Clostridium genera. As such, the bacterial spores may be selected from one or more of Clostridium acetobutylicum, Clostridium butyricum, Clostridium botulinium, Clostridium difficile, Clostridium novyi-NT and Clostridium septicum CS11 (e.g. one or both of Clostridium novyi-NT and Clostridium septicum).

In certain embodiments of the invention, the phosphor material may further comprise an antenna molecule that is complexed to the lanthanide dopant. In other embodiments of the invention that may be mentioned herein, the phosphor material may be one that does not comprise an antenna molecule that can complex to the lanthanide dopant. In embodiments where the antenna molecule is present, it may be selected from one or more of the group consisting of a beta-diketone and triphenylphosphine oxide. In more particular embodiments where the antenna molecule is present, it may be an aromatic beta-diketone.

Any suitable aromatic beta-diketones that can act as an antenna molecule may be used herein. Examples of such molecules includes, but it not limited to, thenoyltrifluoroacetone (TTA), 1-(-2-Naphthoyl)-3,3,3-trifluoroacetone (NTFA), 4,4,4-Trifluoro-1-phenyl-1,3-butanedione (BTFA), and combinations thereof.

In embodiments where the antenna molecule is present, the antenna molecule is complexed to the lanthanide dopant, said complex may have the formula Ln[ANT]ₙ, where Ln is a member of the lanthanide series of elements, ANT is an antenna molecule and n is a whole number integer, optionally wherein n is from 3 to 300, such as 30. In particular embodiments of the invention, Ln[ANT]ₙ may be selected from one or more of the group consisting of Eu[TTA]₃, Sm[TTA]₃, Dy[TTA]₃, Tb[TTA]₃, Eu[BTFA]₃, Eu[NTFA]₃. In more particular embodiments of the invention Ln[ANT]ₙ may be selected from one or more of the group consisting of Eu[TTA]₃, Sm[TTA]₃, and Tb[TTA]₃ (e.g. Eu[TTA]₃, and Tb[TTA]₃, such as and Eu[TTA]₃).

In embodiments where the antenna molecule is present, the antenna molecule is complexed to the lanthanide dopant and suitable molar ratio between these two components may be used. For example, the molar ratio of the lanthanide dopant to the antenna molecule may be from 1:300 to 1:3, such as from 1:200 to 1:10, such as from 1:100 to 1:20, such as about 1:30.

In certain embodiments, the phosphor material disclosed herein may further include a co-dopant material. Any suitable co-dopant material may be used. For example, the co-dopant material may be one or both of Sr²⁺ and Ca²⁺.

The lanthanide dopant may be present in any suitable amount. For example, the lanthanide dopant may be present in an amount of from 20 to 200 nmol per mg dry spore biomass.

As will be appreciated, the phosphor material disclosed herein may be suitable for use in the generation of a material that can be used for various light-emitting applications. As such, in a second aspect of the invention, there is provided a composite material comprising:
a phosphor material as described hereinbefore; and
a bulk material, wherein the bulk material is selected from one or more of the group consisting of a resin, a coating, an adhesive, and an encapsulating material and the bulk material is optically transparent or optically translucent to the excitation and emission wavelengths of the phosphor material.

As noted above, the bulk material may be one of a resin, a coating, an adhesive, and an encapsulating material. As will be appreciated, these materials are, in their final state, intended to be permanently solid at the operating temperature of a suitable system (e.g. an LED), so the operating temperature may be at or close to ambient temperature of may be somewhat higher than ambient temperature. As such, the bulk materials may enable the phosphors to be applied and held in a suitable position for use in a particular device, such as an LED or A LED display. It will be appreciated that the bulk materials can be any suitable material, but it should be a material that will not affect the dormancy of the bacterial spores. As such, it is preferred that the bulk materials do not include water.

The term “optically transparent” refers to a property of a material to allow light to pass through a material without appreciable scattering of light (i.e. on a macroscopic scale, the photons follow Snell's law). As such, the material may be a single component or multiple components that have a uniform index of refraction. In practice, a transparent material may allow light to pass through so that objects behind can be distinctly seen.

The term “optically translucent” refers to a property of a material to allow light to pass through a material, but with an appreciable scattering of light (i.e. on a macroscopic scale, the photons do not follow Snell's law). As such, the material may be formed of a single component with a differential index of refraction or multiple components with different indices of refraction. In practice, a translucent material may allow light to pass through, but objects behind the material may not be distinctly seen.

As will be appreciated, when the bulk material is a single optically transparent material, then the bulk material will be optically transparent. However, if the bulk material is formed from two or more optically transparent materials, then the bulk material may be optically transparent (if the two or more materials have uniform indices of refraction) or it may be optically translucent (if the two or more materials have different indices of refraction).

As noted herein, the bulk material, or bulk materials, may each be a material that is selected to be optically transparent or optically translucent to the excitation and emission wavelengths of the phosphor material. For instance, the Norland 88 adhesive used in the examples section herein was selected because of its UV transparency, as the phosphor materials in said examples have excitation and emission wavelengths in the UV spectrum.

When used herein, the term “encapsulating material” may refer to a material that is intended to fully or partly envelop the phosphor material, and so may act to mechanically protect the phosphor material. Any suitable encapsulation material may be used herein. For example, the encapsulation material may be selected from one or more of the group including, but not limited to, epoxies, epoxy/silicone hybrids, silicones or glasses. In addition to its mechanical protection function, the encapsulation material may reduce light coupling losses and direct a light (e.g. a LED light) towards a specific viewing angle.

As noted above, the composite material (and hence the phosphor material) may be particularly useful in a LED device. As such, in a third aspect of the invention, there is provided a modified light emitting diode, comprising:
a light emitting diode, having a light emitting diode bulb section; and
a cured composite material comprising a phosphor material as described herein and an optically clear adhesive covering a surface of the light emitting diode section, such that light from the light emitting diode passes through the cured composite material.

The light emitting diode may be any suitable light emitting diode. For example, the light emitting diode may be an ultraviolet light emitting diode.

In a fourth aspect of the invention, there is provided a method of forming a phosphor material as described hereinbefore, the method comprising the steps of:
(a) providing a plurality of bacterial spores of the Bacillus and Clostridium genera; and
(b) adding a lanthanide material to the plurality of bacterial spores to provide the phosphor material, wherein the lanthanide material is either a lanthanide salt or a lanthanide complexed to an antenna molecule.

As the components mentioned above have been discussed in relation to the phosphor material hereinbefore a full discussion of these components is omitted here for the sake of brevity.

In the method of forming the phosphor material, the lanthanide material may be provided in any suitable manner. For example, the lanthanide material may be provided in a solvent to the bacterial spores. Any suitable solvent may be used to provide the lanthanide material. For example, the solvent may comprise water and ethanol (e.g. the solvent may be one in which ethanol may form from 1 to 50% vol/vol, such as about 3% vol/vol of the entire solvent (relative to water)). In such embodiments, the lanthanide material may be provided at any suitable concentration. For example, the lanthanide material may be provided in the solvent at a concentration of from 0.005 mM to 10 mM, such as from 0.01 mM to 3 mM, such as from 0.015 mM to 1.5 mM, such as from 0.15 mM to 0.75 mM.

In embodiments of the phosphor material, where an antenna molecule is present, then it may be provided in the solvent for the lanthanides at any suitable concentration. For example, the antenna molecule, when present, may be provided in an amount of from 0.1 mM to 10 mM, such as about 4.5 mM.

As will be appreciated, the process is intended to provide a phosphor having bacterial spores in a dormant state. As such, the method may further comprise a step of substantially removing the solvent from the phosphor material. When used herein, the term “substantially removing” may refer to removing sufficient solvent to prevent the bacterial spore from leaving the dormant state. For example, the solvent remaining may be less than or equal to 5 wt % of the entire spore, such as less than or equal to 2 wt %, such as less than or equal to 1 wt %, such as less than or equal to 0.5 wt %, such as less than or equal to 0.1 wt %, such as less than or equal to 0.01 wt %, such as less than or equal to 0.001 wt % of the entire spore.

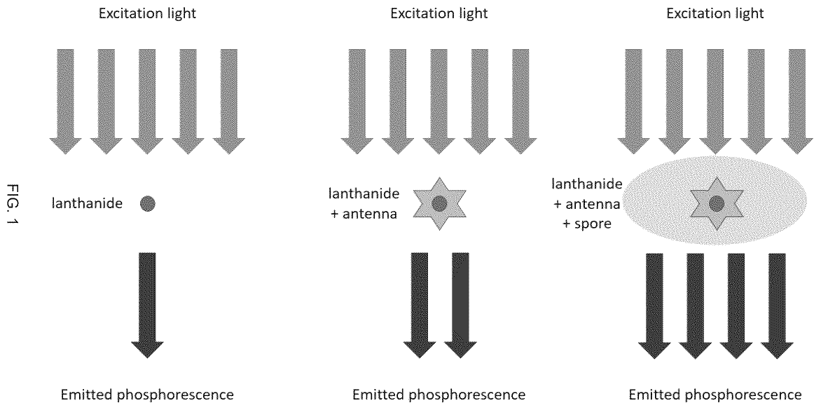

As noted herein, the current invention surprisingly discloses that lanthanides alone or lanthanide antenna complexes may accumulate in spores and turn them into phosphors, and that these phosphors are far more efficient at converting excitatory UV light to emissive phosphorescence than either the lanthanides alone or lanthanide antenna complexes alone. In short, the binding of lanthanides to spores increases their phosphorescent output given the same excitation input, regardless of whether the lanthanide is complexed to an antenna molecule. FIG. 1 describes the general conceptual idea. It is very surprising that the phosphorescence output of the resulting phosphor materials described herein is greater than the situation when the lanthanides (with or without antenna molecules) alone had been excited without the spore.

There are many advantages associated with this invention, some of which are listed below.
(1) Given the environmental impact and potential ecotoxicity of lanthanides, increasing the conversion efficiency of lanthanides would mean that we can achieve the same phosphorescent results with a lower amount of lanthanides.
(2) The process for manufacturing these spore phosphors is simple and just requires a bioreactor to make the spores and admixing the spores with lanthanides ±antenna for instant results. By contrast, doping lanthanides into semiconductor host materials requires clean room facilities and is a ton of technology. For example, at the heart of every LED is a stack of multiple semiconductor layers, each with a different composition and doping concentration. The layers of an LED are deposited using a technology called organometallic vapor-phase epitaxy (OMVPE). Epitaxy ensures that all locations on the wafer have the same atomic orientation and avoids defects that reduce efficiency. Substituting spores for semiconductors would make manufacturing far cheaper and more scalable.
(3) Spores are easily handled and manipulated and can thus be readily incorporated into many materials including resins, coatings, adhesives, composites and casting materials.

In addition, besides being luminescent, one major advantage of spore phosphors is their ease of production. Spores can be scalably produced in bioreactors without the need for clean room or chemical synthesis facilities (Tzeng, Y.-M. et al., J. Appl. Microbiol., 2008, 104, 1275; Zhao, S. et al., World J. Microbiol. Biotechnol., 2008, 24, 2859). Further, spores are extremely monodisperse in size and structure since these parameters are hardcoded into each spore's genetic program. In a sense, nature has had over a billion years to optimize the quality control of spore production. Spores have also been naturally selected for extreme survival, resisting heat, radiation and ultraviolet light for decades, with the boldest clause for the longest-lived bacterial spore being on the order of 10⁸ years (Vreeland, R. H. et al., Nature, 2000, 407, 897). In short, spores are nature's most resilient solution for chemical stability and shelf-life. The practical usefulness of spores as a material is perhaps best demonstrated in its application to self-healing concrete.

Another major advantage of spore phosphors is their flexibility. Combinatorial admixtures of lanthanides, as well as mixing and matching with other antenna molecules and co-dopants, are also trivial to optimise and implement, especially since production of spore phosphors just involves the brief incubation of spores with the materials to be loaded in aqueous solution. Finally, since spores are microparticles and compatible with adhesives, they can be readily incorporated into flexible substrates if required. The combinatorial possibilities are endless.

Various lanthanides and dopants can be arbitrarily incorporated into spores by simple incubation with the spore suspension. The uptake is instantaneous. Dopants like Strontium can further boost the luminescent output of lanthanides.

Further aspects of the invention may be described in the list below.
(1) A luminescent material comprising
   a. a host material which is a bacterial spore
   b. incorporating at least one dopant from the lanthanide series (e.g. Europium)
   c. where the dopant may be complexed with an antenna molecule (e.g. thenoyltrifluoroacetone (TTA))
(2) The composition of (1) where a further co-dopant is added (e.g. Strontium) to enhance the luminescent result.
(3) The composition of (1) where the molar ratio of lanthanide to antenna is carefully calibrated to approximately 1:3 (lanthanide: antenna).

Exemplary embodiments of the invention may make use of Clostridium spp. In addition certain embodiments of the invention (e.g. spores+Eu+TTA) may be especially efficient at converting UV into red light, thereby providing a colour conversion phosphor that may be suitable for use in many applications where for aesthetic or health reasons, ultraviolet (UV) and blue light should be minimized.

In embodiments of the invention that may be mentioned herein, the phosphor material may be one that does not include an antennae molecule.

Further aspects and embodiments of the invention will now be discussed by reference to the following non-limiting embodiments.

### EXAMPLES

### Materials

Europium (III) chloride hexahydrate (212881), Terbium (III) chloride hexahydrate (212903), Dysprosium (III) chloride hexahydrate (289272) and Samarium(III) chloride hexahydrate (204277), TTA or 2-Thenoyltrifluoroacetone (T27006), Strontium chloride hexahydrate (255521), Sodium glycocholate (G7132), Maltose (M5895), Sodium phosphate dibasic (S7907), D-cycloserine (C6880), L-alanine (A7469), L-cysteine (C7352) and Hypoxanthine (H9377), Yttrium oxide phosphor (756490) were purchased from Sigma. DMSO or Dimethyl sulfoxide (D12345) was purchased from Life Technologies. Brain Heart Infusion (BHI) broth (237500), BBL polypeptone peptone (211910) and dehydrated cooked meat media (226730) were purchased from BD Difco. Oxyrase for broth was purchased from Oxyrase Inc and Sigma (SAE0013). Fetal bovine serum (FBS) (S1810) was obtained from iDNA Biotechnology. Percoll (17089109) was purchased from GE Healthcare.

### Bacterial Strains

The following ATCC strains were used: C. novyi (19402), C. septicum (11424) and B. cereus (10987). In the case of C. novyi and C. septicum, non-toxic variants with their alpha toxins genetically inactivated were used. These variants were respectively C. novyi-NT (gift from Professor Bert Vogelstein's lab, The John Hopkins University, Baltimore, Maryland, USA) and C. septicum CS11 (derived from ATCC 11424 by inactivating its lethal alpha toxin for safe handling). B. subtilis 168 wild-type (P1A1), B. megaterium (7A16 i.e. QM B1551) and B. thuringiensis(4AJ 1 ) were purchased from the Bacillus Genetic Stock Centre (BGSC), USA. Stock solutions were prepared in Luria-Burtani (LB)-Glycerol mixture (30% glycerol) and stored at −80 °C.

### Example 1. Spores Sequester the Lanthanide Europium

To develop efficient spore based host lattices for lanthanide phosphors, we first wanted to identify the best spore system by quantifying the lanthanide uptake by different bacterial spores. For this, we conducted a screen using two non-pathogenic bacteria each from the spore forming Bacilli (Bacillus subtilis, Bacillus thuringiensis) and Clostridia (Clostridium novyi-NT, C. septicum—CS11) genera. Since most bioremediation studies have been conducted using the vegetative form in bacteria (Moriwaki, H. et al., Appl. Microbiol. Biotechnol., 2013, 97, 3721; Fischer, C. B. et al., RSC Adv., 2019, 9, 32581; Moriwaki, H. & Yamamoto, H., Appl. Microbiol. Biotechnol., 2013, 97, 1), we tested each bacteria using their spore forms (both dormant and germinated) as well as their vegetative forms. Europium (Eu) as a model lanthanide was tested both alone and in combination with the beta-diketone thenoyltrifluoroacetone (TTA). TTA was included because we had shown in a previous Raman spectroscopy study that it enhanced the localization of Eu to C. novyi-NT spores (Singapore patent application no. 10202260603W). Eu uptake was inferred by using ICP-OES to measure the amount of Eu remaining in solution after incubation with spore or vegetative forms. The Eu concentration chosen (0.14 mM) was within the typical range used in other bioremediation/lanthanide uptake studies. The TTA concentration used (4.2 mM) was thirty times greater than that of Eu.

Clostridium novyi-NT Sporulation

Spores of Clostridium novyi -NT strains were generated according to the method reported by Cheong et al. (Cheong, I. et al., Science, 2006, 314, 1308). Briefly, 200 μL of 5×10⁹ CFU/mL of C. novyi-NT spores were inoculated anaerobically in a GasPak™ anaerobic jar at 37 °C, pH 7.4 in 1 L of medium containing 5 g Na₂HPO₄, 30 g polypeptone peptone, 0.5 g L-cysteine, 10 g maltose, 50 g dried cooked meat particles (Difco) and 10% v/v FBS after 3 weeks in this medium, spores were purified from contaminating vegetative forms on a discontinuous Percoll (90%) gradient at 15000 rcf for 30 minutes in a Beckman Avanti J-20 XP high performance centrifuge. The quality of spores was confirmed by phase contrast microscopy and contained >99% phase bright spores. The spore concentration was adjusted approximately to the order of 5×10⁹ CFU/mL by appropriate dilution in 1X PBS (phosphate-buffered saline) using a standard curve correlating OD600 absorbance to cell count.

### Bacillus Sporulation

For B. subtilis, B. cereus, B. megaterium and B. thuringiensis spores, sporulation was done based on the protocol from Nicholson and Setlow (Nicholson W. L. and Setlow, P. Sporulation, germination and outgrowth. in Molecular Biology Methods for Bacillus (eds. Harwood, C. R. & Cutting, S. M.) 391-450 (John Wiley & Sons, 1990)). Bacillus strains were inoculated on a Luria Bertani (LB) agar plate overnight at 37 °C. The next day, single colonies were inoculated into 2xSG medium (Nicholson W. L. and Setlow, P. Sporulation, germination and outgrowth. in Molecular Biology Methods for Bacillus (eds. Harwood, C. R. & Cutting, S. M.) 391-450 (John Wiley & Sons, 1990)) (30 mL, pH 7) for all the other Bacilli except for B. megaterium for which supplemented nutrient broth (SNB) medium at pH 7.2 was used. These were then allowed to grow on a MaxQ 8000 Orbital shaker (200 rpm, 37 °C) till it reached an OD600 value of 0.3-0.5. This culture (25 mL) was then re-inoculated in a 2 L Erlenmeyer flask containing 2xSG media or SNB media respectively (225 mL) and covered with a gas permeable membrane (Breathe Easy®) for allowing sufficient aeration. The flask was then incubated in a Gerhardt orbital shaker (130 rpm, 37 °C) for 92 hours. The harvested spores were washed in 1× PBS followed by isopycnic centrifugation using self-forming Percoll gradient (90%) in a Beckman Avanti J-20 XP high performance centrifuge (JS 13.1 rotor, 15000 rcf, 30 min, 4 °C). The majority of the phase-bright spores were obtained as the bottom fraction for the strains except for B. cereus where the spores were obtained as the top fraction. These fractions were repeatedly washed with 1X phosphate buffer saline (PBS) and stored in 4 °C until further use. The quality of spores was confirmed by phase contrast microscopy and contained >99% phase bright spores. The spore concentration for all strains was counted using a Neubauer counting chamber (Petroff Marenfield) with special depth and was adjusted approximately to the order of 10⁹ CFU/mL for each strain.

Clostridium septicum Sporulation

C. septicum CS11 spores were purified according to the protocol by Dang et al (Dang, L. H. et al., Proc. Natl Acad. Sci. USA, 2001, 98, 15155), with some modifications. All steps were performed inside a Plas labs anaerobic chamber. Briefly, overnight cultures of C. septicum were diluted 50× into 100 mL of BHI-S media containing 0.05% L-Cysteine and incubated until the OD600 was 1.5-3 before the entire culture was added into 900 mL of sporulation media (0.05% L-cysteine, 3% bacto polypeptone, 5% dehydrated cooked meat medium, and 10% fetal bovine serum) and incubated at 37 °C for five days. Spores were purified from vegetative cells on an 80% discontinuous Percoll gradient at 15000 rcf for 30 minutes in a Beckman Avanti J-20 XP high performance centrifuge. The obtained spores were washed twice, resuspended in water and stored at 4 °C until further use. Spore quality was checked by phase contrast microscopy and was found to contain >99% phase bright spores. The spore concentration for all strains was counted using a Neubauer counting chamber (Petroff Marenfield) with special depth and was adjusted approximately to the order of 10⁹ CFU/mL for each strain.

### Inductively Coupled Plasma Optical Emission Spectroscopy (ICP-OES) Sample Preparation

Dormant spores in 1× PBS were used at the order of 10⁹ CFU/mL, slightly concentrated and re-suspended in saline solution (150 mM NaCl).

Germination of spores was performed as follows: C. novyi-NT spores were germinated in L-cysteine (100 mM), hypoxanthine (0.1 mM) and oxyrase mixture (1:50) while C. septicum spores were germinated in sodium glycocholate (61 mM) and oxyrase mixture (1:14). B. subtilis and B. thuringiensis spores were first heat activated at 70 °C in 1× PBS (1 hour) and (7.3 mM) D-Cycloserine solution (30 min) respectively, followed by germination in L-alanine (74 mM). All germination experiments were done at 37 °C for 30 minutes except for C. septicum for 3 hours. Following this, the spores were re-suspended in saline solution (150 mM NaCl).

For vegetative bacteria samples, overnight cultures of B. subtilis and B. thuringiensis were grown in LB media at 37 °C and 220 rpm. C. novyi-NT was grown in BHI-10%FBS media in the presence of oxyrase at 37 °C while C. septicum was grown in BHI-10% FBS inside a Plas labs anaerobic chamber at 37 °C. The vegetative bacteria were harvested approximately after 20 hours and re-suspended in saline solution (150 mM NaCl).

Equal volumes of europium chloride (0.15 mM) and TTA (4.5 mM) in 3% ethanol were mixed and incubated at room temperature for 30 minutes before use.

To 35 μL of spores/vegetative bacteria, 500 μL of Eu or Eu-TTA were added such that final concentrations were 0.14 mM (Eu), and 4.2 mM (TTA) respectively. The samples were immediately centrifuged at 3900 rcf, 5 minutes followed by centrifugation at 15000 rcf, 5 minutes on a Thermo Scientific Sorvall Legend Micro 17 centrifuge. The supernatants (500 μL) from these solutions were diluted to 10 mL using 3% ethanol followed by addition of 221 μL of nitric acid. These samples were then analysed using a Perkin Elmer Avio 500 ICP-OES machine to determine the Eu concentration (ppm) remaining in the supernatant. Eu uptake by the spore/vegetative bacteria was determined by deducting this supernatant concentration from the starting Eu concentration of 0.14 mM.

For biomass determination, equal volumes of spore or vegetative bacteria suspensions (35 μL) were added to a preheated (60 °C, 4 hours) empty 1.5 mL Eppendorf tube and dried overnight in a Sanyo MOV 112 drying oven set to 60 °C temperature. The biomass weight for spores and vegetative bacteria was determined by measuring the weight difference between the respective dried biomass containing Eppendorf tube and the empty Eppendorf tube prior to addition of the biomass, using a Mettler Toledo AT261 Delta range weighing balance. Final biomass values used were an average of three independent samples per bacterial form.

### Results and Discussions

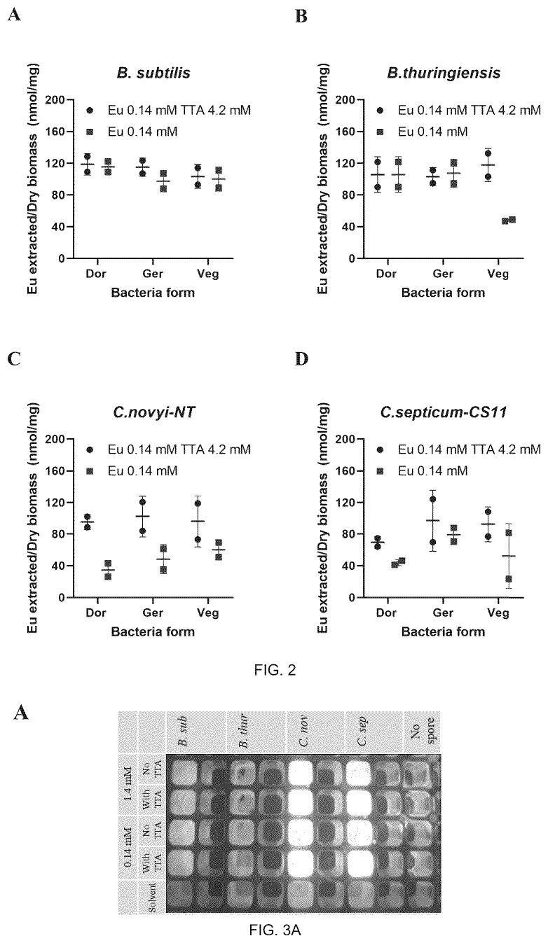

Major differences were observed in Eu uptake between bacteria, between bacterial forms and also between Eu with and without TTA (FIG. 2). To an approximation, both dormant and germinated spores performed on par with vegetative forms, with the exception of B. thuringiensis where Eu uptake without TTA was much poorer for vegetative forms than for spores. The implication is that both dormant and germinated spores are good substitutes for vegetative forms when it comes to Eu uptake.

Looking only at the spore forms, Bacillus spores performed better than Clostridium spores in uptake of Eu alone. Both Bacillus spores had similar levels of uptake at around 100-140 nmol/mg spore, regardless of whether TTA was present (FIG. 2A and FIG. 2B). These uptake levels were in the same range as an earlier study showing uptake of other lanthanides like Terbium and Dysprosium for dormant B. subtilis spores (Fischer, C. B. et al., RSC Adv., 2019, 9, 32581; Dong, W. et al., Appl. Environ. Microbiol., 2019, 85, e00956-19). In contrast, Clostridium spores took up much less Eu (<50 nmol/mg) (FIG. 2C and FIG. 2D). The addition of TTA however improved the performance of Clostridium spore Eu uptake to be almost on par with Bacillus spores. TTA's presence especially boosted Eu uptake in C. novyi-NT by threefold. We speculate that this differential effect of TTA on spore Eu uptake might be due to structural differences between Bacillus and Clostridium spores. Whether spores were dormant or germinated had minimal effect on the result except for C. septicum where germinated spores had approximately twice the Eu uptake of dormant spores.

We concluded that both B. subtilis and B. thuringiensis spores were highly efficient in lanthanide sequestration regardless of TTA's presence while the Clostridium spores required the TTA to achieve comparable levels of lanthanide uptake. We proceeded to use dormant spores for the rest of our experiments (as demonstrated in the other Examples). Compared to germinated spores and vegetative forms, they are structurally more robust and perform equally well in lanthanide sequestration. Further, once prepared by sporulation, dormant spores are stored ready for use without further steps.

### Example 2. Europium-loaded Spores Display Phosphorescent Properties

We wondered if lanthanide uptake by spores would translate to luminescence when the spore was used as a phosphor. Bacillus and Clostridium dormant spores were obtained using the protocol described in Example 1.

Bacillus and Clostridium dormant spores were loaded with Eu (0.14 mM or 1.4 mM) with or without TTA, and dried overnight under vacuum in a 384 microwell plate after which luminescence was observed and imaged.

### Spore Phosphor Preparation

For Eu based phosphors, equal volumes of europium chloride (3 mM, 0.3 mM or 0.03 mM) and TTA (9 mM) in 3% ethanol were mixed and incubated at room temperature for 30 minutes before use. To 35 μL of dormant spores or saline, 500 μL of Eu or Eu-TTA were added such that final concentrations were 1.4 mM, 0.14 mM or 0.014 mM (Eu), and 4.2 mM (TTA) respectively.

For other lanthanide phosphor experiments, equal volumes of samarium chloride or terbium chloride or dysprosium chloride (3 mM or 0.3 mM) and TTA (9 mM) in 50% ethanol were mixed and incubated at room temperature for 30 minutes before use. To 35 μL of dormant spores or saline, 500 μL of Sm/Tb/Dy or Sm/Tb/Dy-TTA were added such that final concentrations were 1.4 mM or 0.14 Sm or Tb or Dy), and 4.2 mM (TTA) respectively.

The samples were immediately centrifuged at 3900 rcf, 5 minutes followed by centrifugation at 15000 rcf, 5 minutes on a Thermo Scientific Sorvall Legend Micro 17 centrifuge. The supernatants (500 μL) from these solutions were discarded and the remaining 35 μL pellets were transferred either to PCR tubes or to a Greiner Bio-one 384-well UV star microtiter plate. These pellets were then dried overnight under high vacuum.

Visualisation of Spore Phosphor Fluorescence Spore phosphor fluorescence was either visualized using a Major Science MUV26 series UV transilluminator (254 nm and 365 nm excitation wavelengths) or measured using a Tecan Spark® multimode fluorescence microplate reader at 370 nm excitation and 620 nm emission (for Eu based spore phosphor) with a lag time of 20 μs, Gain 50, Integration time 1000 μs unless specified.

### Excitation-Emission Matrix (EEM) Measurement

For EEM measurements, an excitation range of 250-400 nm and emission range of 450-800 nm with a lag time of 20 μs, Gain 50, Integration time 1000 μs were used. EEM data were converted to a log₁₀ scale in Microsoft Excel (negative and zero intensity values were converted to 1 before performing the logarithmic conversion) and contour plots were plotted using the Plotly package in Rstudio (2022.07.2, Build 576).

All other graphs were created using Graphpad Prism 9.

### Results and Discussions

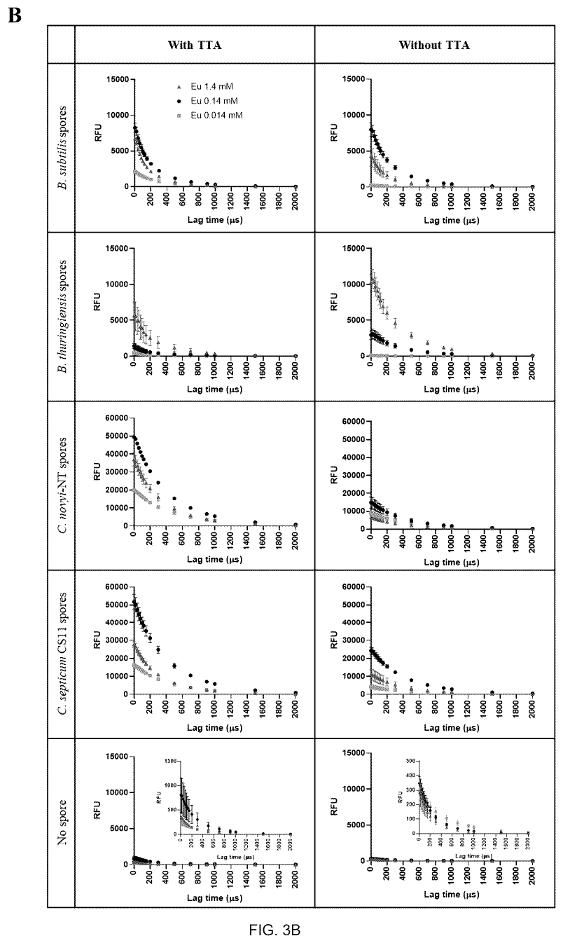

In this study, different bacterial spores loaded with the same Eu+TTA activator gave vastly different results. When illuminated under a 365 nm UV transilluminator, all four bacterial spores showed visible luminescence compared to the no spore controls (which were Eu at 0.14 mM and 1.4 mM with or without TTA) (FIG. 3A). There was no visible difference in brightness between Eu at 0.14 mM and 1.4 mM. Intriguingly, the Bacillus spores showed far less luminescence compared to the Clostridium spores. This shows that increased Eu uptake does not equate to higher luminescence output. It again suggests that differences in composition and architecture of the spores are as equally important as Eu uptake. Between the Bacillus spores, B. subtilis exhibited higher fluorescence compared to B. thuringiensis.The luminescence of the Clostridium spores was too bright to resolve any differences between C. novyi-NT and C. septicum. The Bacillus spores were not as bright as the Clostridium spores even though they had higher Eu uptake.

To quantify the luminescence of these spore phosphors, we loaded spore phosphors at three Eu levels (0.014mM, 0.14 mM, 1.4mM) with or without TTA, dried them under vacuum, and measured their time-resolved luminescence output (excitation: 370 nm, Emission: 620 nm) as lag time increased (FIG. 3B). All spore phosphors produced substantial luminescence output in comparison with the no spore control. When considering the best performing luminescence curve for each spore phosphor, luminescence output was still measurable after 500us. This long temporal decay is consistent with the luminescence output, which exhibited a phosphorescent rather than fluorescent character. In all samples tested, Eu at 0.014 mM produced the lowest luminescence as expected. However, in all the spore phosphors except B. thuringiensis, Eu at 0.14 mM produced greater luminescence than Eu at the highest concentration of 1.4 mM. We think that the reduction in luminescence reflects quenching at higher concentrations of Eu within the spores. The addition of TTA increased the luminescence of the spore phosphors except in B. thuringiensis and to a lesser extent in B. subtilis. The quantitative luminescence decay data corroborated our initial visual inspection of the spore phosphors, and emphasized that the level of luminescence output is heavily dependent on the bacteria used.

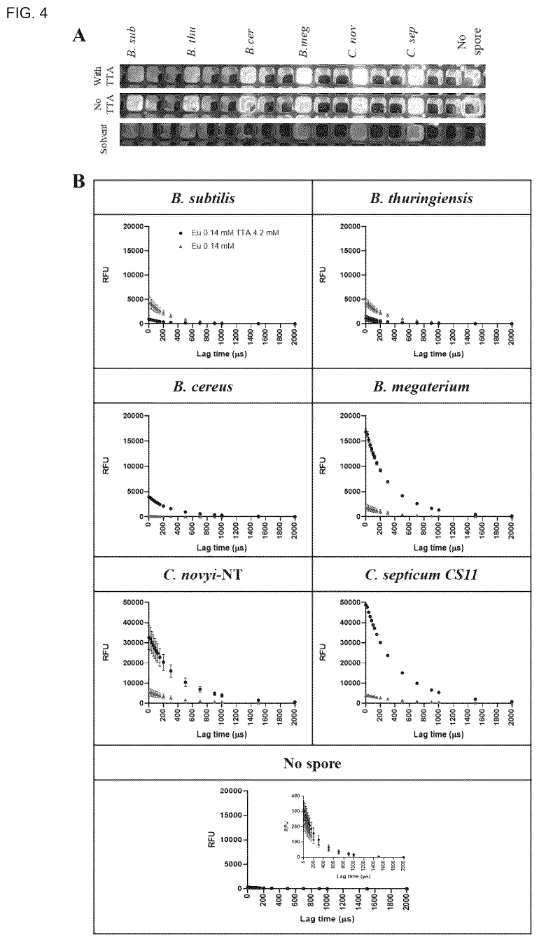

Since the two Bacillus spores tested (B. subtilis and B. thuringiensis) did not show significant luminescence, we expanded our screening panel to include two more Bacillus strains to test if the spore luminescence observed is restricted only to Clostridium.For this, we prepared B. cereus and B. megaterium spore phosphors with Eu (0.14 mM) and compared their luminescence with the other spores in the earlier experiment (FIG. 4A). To our surprise, both B. cereus and B. megaterium spores showed significant luminescence in the presence of TTA compared to B. subtilis and B. thuringiensis spores. Even though being slightly lower than the Clostridium spores, B. megaterium spores in particular showed bright red fluorescence. In the absence of TTA, B. megaterium spores showed luminescence comparable to the Clostridium spores. Quantitatively, B. megaterium spores exhibited almost a 15-fold increase in luminescence in the presence of TTA when compared to B. subtilis or B. thuringiensis (FIG. 4B). B. megaterium spores also showed measurable phosphorescence beyond 500 μs, which is similar to the other spores. These results thus indicate that spore phosphors can be prepared using both Clostridium and Bacillus spores.

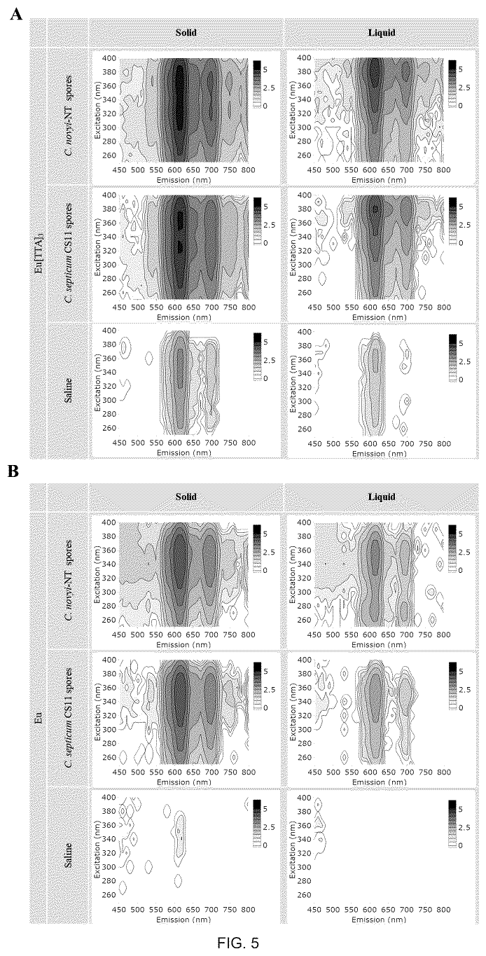

Lanthanide phosphorescence is known to be affected by vibrational coupling in aqueous environments (Werts, M. H. V. Sci. Prog., 2005, 88, 101; Meshkova, S. B. et al., J. Appl. Spectrosc., 1997, 64, 229). Hence, the presence or absence of water could theoretically affect the absorption and emission profiles of the spore phosphors. C. novyi-NT and C. septicum had the highest luminescence output and were hence used to study this question. Spores were first loaded with Eu (1.4 mM) with or without TTA (4.2 mM), then they were either dried solid or left in aqueous liquid suspension. Spectral scans were performed to obtain excitation-emission matrix (EEM) profiles of these spore phosphors (FIG. 5A and FIG. 5B). The data reveals that for all spore phosphors tested, dried spore phosphors showed EEM profiles which were visibly different from spore phosphors in aqueous suspension. The emission spectra predominantly consists of two maxima peaks at 610/620 nm corresponding to the 5D₀-⁷F₂ transition, and a smaller peak at 700 nm corresponding to the ⁵D₀-⁷F₄ transition for Eu. The stronger emission peaks for the solid condition compared to the liquid condition is likely attributable to the removal of solvent effects in the solid condition. Perhaps the most distinctive difference between the solid and liquid profiles was in the excitation spectra. While the spores for the liquid condition were excitable only within 360-380 nm, the solid spore phosphors were excitable within a far broader range of 300-380 nm (FIG. 5A and FIG. 5B). This implies that even though spores can perform as phosphors while in aqueous suspension, eliminating water will still increase luminescence output by increasing the spectral range of ultraviolet light that can be captured by the spore phosphor. The addition of TTA did not cause any qualitative change in the EEM spectra in both states and only led to a quantitative enhancement of luminescence.

### Example 3. Long-term Stability of Spore Phosphors

Next, we evaluated the long-term stability of spore phosphors. Spore phosphors were prepared by following protocols in Examples 1 and 2. Spores were loaded with Eu (1.4 mM and 0.14 mM) with or without TTA (4.2 mM), then vacuum-dried till solid in 384-well microtiter plates. Plates were stored on the bench with non-airtight lids in ambient air and room temperature. Time-resolved luminescence (Ex: 370 nm, Em: 620 nm, lag time 0.02 ms) was measured over the course of 24 weeks.

### Results and Discussions

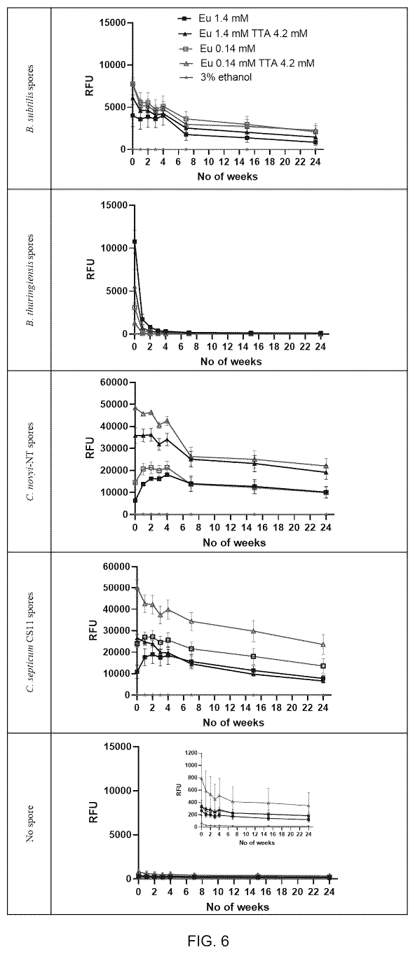

B. thuringiensis showed a dramatic drop in luminescence after just the first week. The other three bacteria in contrast, produced luminescence substantially higher than the no spore lanthanide alone control for the 24-week period (FIG. 6). They also seemed to stabilise in luminescence output after an initial 7-week period where output was highly variable. Among the tested bacteria, C. novyi-NT seemed to have the best profile of high and stable luminescence output over the 24 weeks. It is likely that incorporating the spore phosphors into materials which exclude moisture and oxygen will further improve their long-term stability.

### Example 4. Varying the Lanthanides to Produce Different Colours

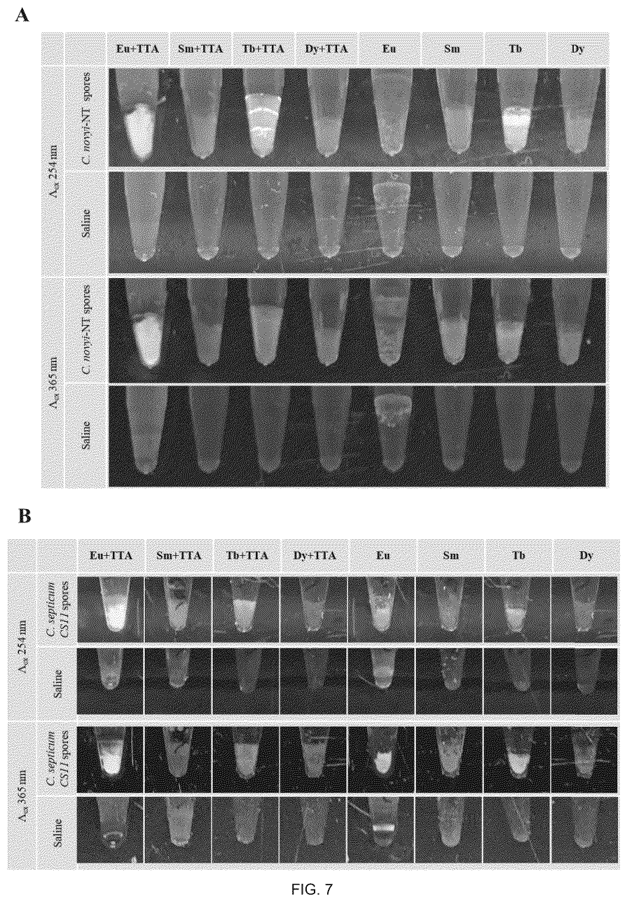

While the use of Eu as an activator creates spore phosphors with orange red emission, we are not limited to Eu since other colour outputs are possible by varying the lanthanide loaded into the spore. To see if this was true, Eu, as well as Samarium (Sm), Terbium (Tb) and Dysprosium (Dy), were loaded either with or without TTA into C. novyi-NT or C. septicum spores by following protocol disclosed in Example 2. The spores were then dried to generate a panel of spore phosphors, then visualised under a UV transilluminator set at either 254 nm or 365 nm (FIG. 7A and FIG. 7B).

### Results and Discussions

Differences were observed in the luminescence output which were consistent with the expected emission spectra for Sm and Tb: Sm spore phosphors appeared to be red while Tb spore phosphors appeared to be green. In contrast, luminescence from Dy spore phosphors was not visible in both bacteria, regardless of the excitation wavelength and the presence or absence of TTA. It is likely that further optimization will be required to observe luminescence with Dy-loaded spores.

Just like for Eu, the addition of TTA made a dramatic difference for Sm spore phosphors (FIG. 7A and FIG. 7B). Spores loaded with Sm were barely luminescent whereas the addition of TTA improved luminescence output for both bacteria (except at 254 nm for C. septicum spores where both Sm and Sm+TTA loaded spores did not show luminescence). Tb's luminescence profile differed from Sm and Eu. TTA did not improve Tb's green luminescence output for C. novyi-NT regardless of excitation wavelength. Likewise, for C. septicum spores excited at 365 nm, TTA was associated with dramatically decreased luminescence while TTA did not seem to have any improved effect on luminescence at 254 nm. This data emphasizes again that the choice of bacteria and antenna molecule has huge effects on luminescence output for spore phosphors.

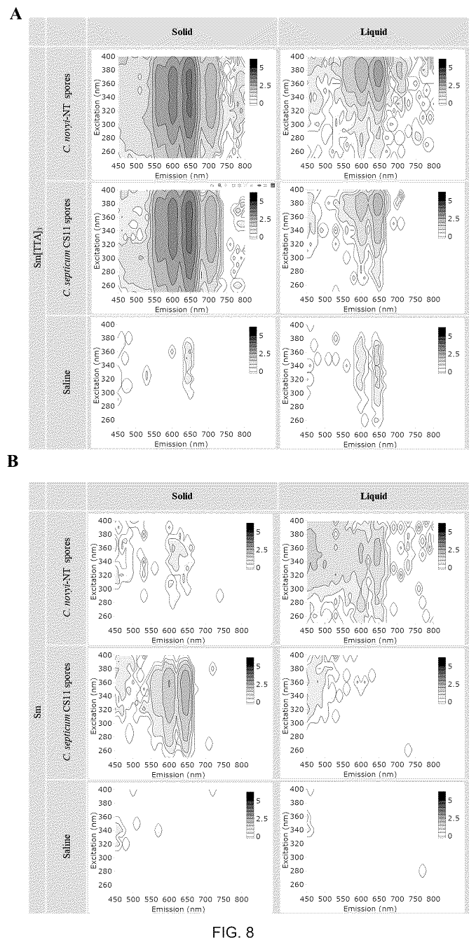

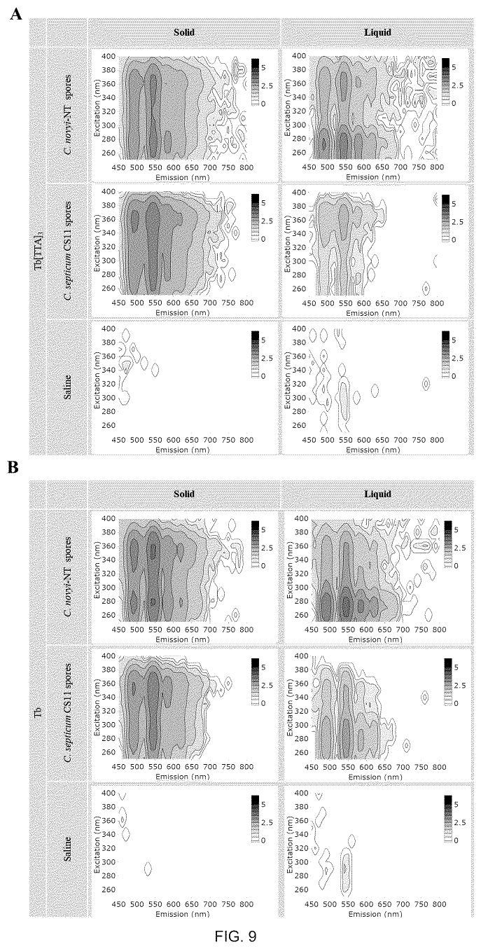

The Sm and Tb phosphors were studied in further detail by generating their excitation emission matrices (EEM). Spores loaded with Sm and TTA showed red fluorescence with emission maxima at 560 nm ⁴G_{5/2}-⁶H_{5/2}, 600 nm ⁴G_{5/2}-⁶H_{7/2}, 650 nm ⁴G_{5/2}-⁶H_{9/2} and 700 nm ⁴G_{5/2}-⁶H_{11/2}. The solid form exhibited a broader excitation range extending into the UV-B region, resulting in a 10 to 100-fold increase in emission intensity compared to its liquid form (FIG. 8A). Sm spore luminescence was far weaker when TTA was omitted (FIG. 8B). As shown in the visual data, Tb performed quite differently from Sm and Eu (FIG. 9A and FIG. 9B). The excitation range was generally broad for Tb loaded spores, regardless of bacteria, state (solid vs liquid) or presence of TTA. Tb-loaded spores exhibited green fluorescence with emission maxima at 480 nm ⁵D₄-⁷F₆, 545 nm ⁵D₄-⁷F₅, 580 nm ⁵D₄ -⁷F₄ and 620 nm ⁵D₄-⁷F₃. Also relevant is the observation that TTA makes a difference in enhancing luminescence for Eu but not necessarily for Tb. This was observed especially for the case of C. septicum spores where Tb alone outperformed Tb+TTA.

The high conversion efficiency from UV to red means that this invention can convert a lot more of the UV light from the LED semiconductor element than just the lanthanide alone. There is currently an issue with UV/blue leakage from LEDs impacting human circadian rhythms (i.e. sleep cycles). This invention has the potential to be a colour conversion phosphor to address this issue.

### Example 5. Co-doping with Strontium for Further Increased Luminescence

With many rare earth-based phosphors, co-doping with other metal ions often enhances luminescence (Lin, Y. et al., J. Eur. Ceram. Soc., 2003, 23, 175; Santos, P. C. L. et al., Phys. Status Solidi B Basic Res., 2019, 256, 1900024). We hence wondered if similar enhancement would work with spore phosphors. Here, we co-doped C. novyi-NT spores with strontium chloride (476 mM), TTA (5.4 mM) and Eu (either 0.06 mM or 1.9 mM). Spores were obtained by using the protocol described in Example 1.

### Strontium Doping of Spore Phosphor

Appropriate volumes of europium chloride (3.9 mM or 0.12 mM in DIW), strontium chloride (1 M in DIW), TTA (225 mM in DMSO) and C. novyi-NT dormant spores in 1× PBS were mixed such that final concentrations of 1.9 mM or 0.06 mM (Eu), 476 mM (Strontium chloride) and 5.4 mM (TTA) were obtained. In the control sample, strontium chloride was replaced with DIW. The samples were immediately centrifuged at 3900 rcf, 5 minutes followed by centrifugation at 15000 rcf, 5 minutes on a Thermo Scientific Sorvall Legend Micro 17 centrifuge. The supernatants (1 mL) from these solutions were discarded and the remaining 50 μL pellets were transferred either to PCR tubes or to a Greiner Bio-one 384-well UV star microtiter plate. These pellets were then dried overnight under high vacuum.

Strontium doped spore phosphor fluorescence was either visualized using a Major Science MUV26 series UV transilluminator (365 nm excitation wavelengths) or measured using a Tecan Spark® multimode fluorescence microplate reader at 370 nm excitation and 620 nm emission, Gain 40, integration time 1000 μs and varying lag times.

### Results and Discussions

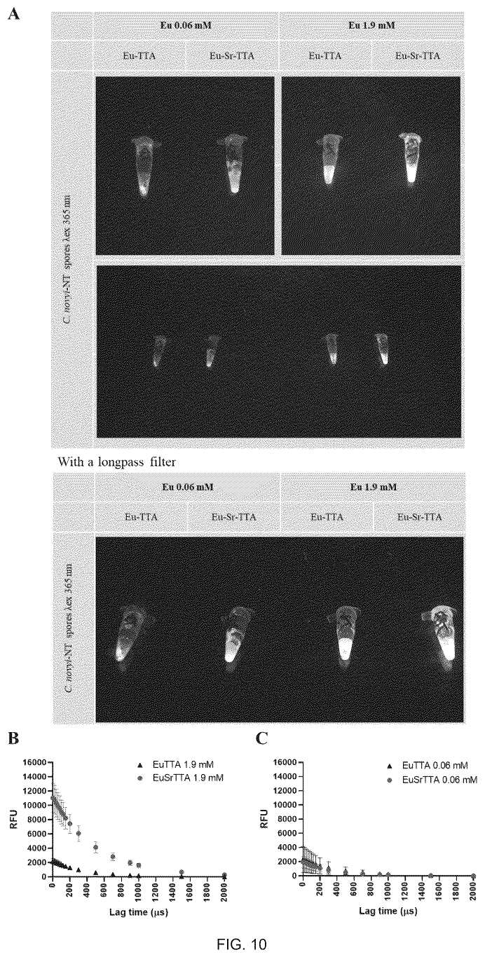

Addition of strontium visibly increased the luminescence of Eu+TTA spore phosphors as visualised using UV transillumination at 365 nm (FIG. 10A). Intensity enhancement was more apparent at the higher Eu concentration (1.9 mM). At this higher concentration, the luminescence of Eu-Sr-TTA doped spore phosphors nearly increased five-fold in intensity compared to equivalent spore phosphors without Sr co-doping (FIG. 10B). At a much lower concentration of Eu 0.06 mM, doping of Sr did not produce any appreciable difference in luminescence (FIG. 10C). The possibility of co-doping various metals into spore phosphors just by brief incubation creates endless combinatorial possibilities for tuning the performance of spore phosphors.

### Example 6. Comparison of Spore Phosphors with Commercial Yttrium Oxide Phosphor

To benchmark our spore phosphors, we compared their luminescence against Y_{1.98}Eu_{0.08}O₃, a commercially available red phosphor. Yttrium (Y) phosphors are the most commonly used red phosphors and are often found in television screens owing to their narrow band emission around 610 nm (Rao, R. P., Solid State Commun., 1996, 99, 439). We prepared C. novyi-NT spores loaded with Eu and TTA, as well as Y phosphors containing an equivalent quantity of Eu. Spores were obtained by using the protocol described in Example 1.

Comparison with Commercial Yttrium Oxide Phosphor

Yttrium oxide powder was suspended in DIW at an Eu concentration of 0.14 mM. The same was added to create the Eu based spore phosphors. This suspension was then dried overnight under high vacuum to obtain the solid yttrium oxide phosphor. The yttrium oxide water suspension was used as the liquid sample. Eu (0.14 mM)-TTA (4.2 mM) doped C. novyi-NT spore phosphor was used as the spore phosphor in both liquid and solid forms. Equal volumes of the yttrium oxide phosphor and spore phosphors were used for drying and in the liquid form. Phosphor fluorescence was either visualised using a Major Science MUV26 series UV transilluminator (254 nm and 365 nm excitation wavelengths) or measured using a Tecan Spark fluorescence microplate reader at 370 nm or 250 nm excitation and 620 nm emission with no lag time, gain 50, integration time 1000 μs.

### Results and Discussions

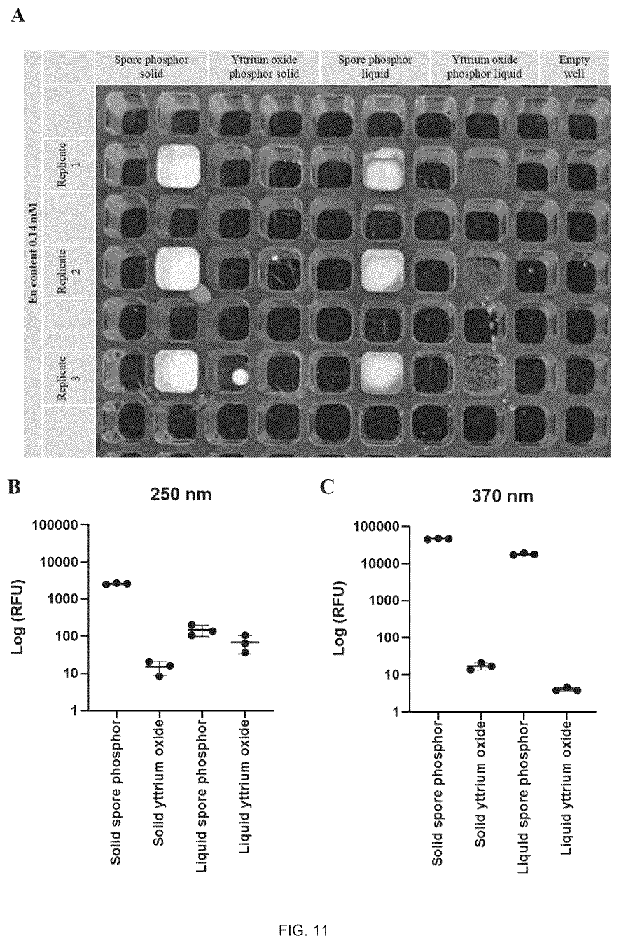

Under UV transillumination (254 nm), spore phosphors were brighter than Y phosphors, regardless of whether they were dried solids or in liquid suspension (FIG. 11A). In relative terms, spore phosphors were brighter as a dried solid than in liquid suspension, but Y phosphors showed the opposite trend and were brighter in liquid suspension than as a solid. To quantify these observations, fluorescence was measured both at 250 nm and 370 nm since these are the respective excitation maxima for the Y phosphor and the spore phosphor respectively. Consistent with the visual results, Y phosphors excited at their optimal excitation (250 nm) were less bright than spore phosphors excited at the same wavelength (FIG. 11B). Using the optimal excitation (370 nm) exaggerated this gap even further, such that spore phosphors were 3 logs brighter than Y phosphors (FIG. 11C). Spore phosphor shows 1000-fold increase in fluorescence compared to commercial yttrium oxide phosphor. These results demonstrate that spore phosphors can have superior quantum efficiency compared to commercial Y phosphors.

This invention has high quantum efficiency and is cheap to manufacture which will allow us to use this rare earth element resources more efficiently and to minimize negative impacts on the environment.

### Example 7. Spore Phosphor Coated LEDS for Color Tunability

One of the most essential applications of phosphors is their use in LEDs. One key question in this application is how effective the phosphor is in colour conversion (Xia, Z. & Liu, Q., Prog. Mater. Sci., 2016, 84, 59). Besides the aesthetic goal of achieving a desired emission profile, there is also the concern that UV and blue light can have undue effects on human circadian rhythms. For example, melatonin is produced by the pineal gland in the brain and is responsible for regulating sleep. Its production increases in darkness, but exposure to light, especially UV and blue light, can suppress melatonin production, hence disrupting sleep patterns.

We wondered how effective spore phosphors would be in converting UV light from an LED into red light. Two forms of UV LEDs were used. The first was individual LED bulbs with emission at 365 nm. These were well suited for Eu excitation. The second was an LED bulb strip emitting at 275 nm which was more appropriate for Tb excitation. C. novyi-NT spore phosphors were mixed into a UV-transparent adhesive (Norland 88) and coated on either of the UV LEDs.

Spore Phosphor Coated LED Preparation Spore phosphors were prepared by following the protocol in Example 2. Solid spore phosphors (0.14 mM lanthanide concentration—TTA 4.2 mM) prepared in a PCR tube were mixed with a drop of Norland 88 adhesive and coated on top of a commercially available UV LED bulb using a pipette tip. The coated LEDs were cured for 10-15 minutes under a 365 nm UV transilluminator. The coated LED bulbs were then powered by a single GP Ultra plus alkaline 9V battery or multiple 9V batteries in series connected on a breadboard and images were taken using a Nikon COOLPIX L120 DSLR camera. For Eu based spore phosphors, UV-A LED bulbs with 365 nm excitation (EOLD- 365-525 LED, element 14) were used while for Tb based spore phosphors, UV-C LED bulbs with 275 nm excitation (ILS-OV12-O275-VL004-SC201-W2, element14) were used.

### Results and Discussions

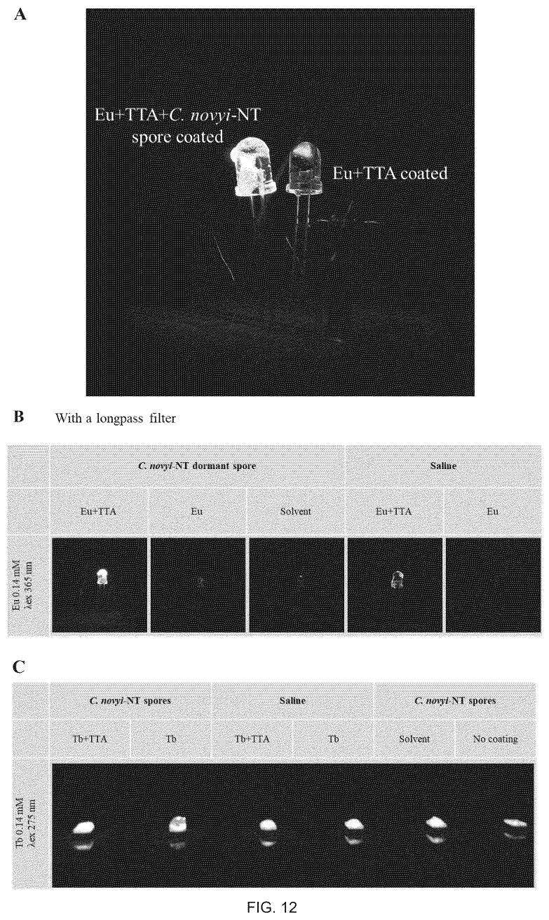

The results were illuminating. The individual UV LED coated with Eu+TTA spore phosphor emitted red orange luminescence which was visibly much brighter than just Eu+TTA alone, or any of the other controls (FIG. 12A and FIG. 12B). A more subtle result was obtained with the UV LED strip bulb coated with Tb+TTA spore phosphors, which produced a slight greenish tinge compared to controls which were faint blue owing to the UV excitation (FIG. 12C).

1. A phosphor material comprising:
a bacterial spore as a host material; and
a lanthanide dopant within the host material and/or on a surface of the host material, wherein:
the bacterial spore is in a dormant state; and
the bacterial spore is selected from one or more bacterial spores selected from one or more of the Bacillus and Clostridium genera.

2. The phosphor material according to claim 1, wherein the lanthanide dopant is selected from one or more of the group consisting of Eu³⁺, Sm³⁺, Tb³⁺, and Dy³⁺, optionally wherein the lanthanide dopant is selected from one or more of the group consisting of Eu³⁺, Sm³⁺, and Tb³⁺ (e.g. Eu³⁺).

3. The phosphor material according to claim 1, wherein the bacterial spores are formed from one or more of Bacillus megaterium, Bacillus cereus, Bacillus subtilis, Clostridium acetobutylicum, Clostridium butyricum, Clostridium botulinium, Clostridium difficile, Clostridium novyi-NT and Clostridium septicum CS11, optionally wherein the bacterial spores are formed from one or more of Bacillus megaterium, Clostridium novyi-NT and Clostridium septicum (e.g. one or both of Clostridium novyi-NT and Clostridium septicum).

4. The phosphor material according to claim 1, wherein the phosphor material further comprises an antenna molecule that is complexed to the lanthanide dopant.

5. The phosphor material according to claim 1, wherein the antenna molecule is selected from one or more of the group consisting of a beta-diketone and triphenylphosphine oxide.

6. The phosphor material according to claim 1, wherein the antenna molecule an aromatic beta-diketone, optionally wherein the aromatic beta-diketone is selected from one or more of the group consisting of thenoyltrifluoroacetone (TTA), 1-(-2-Naphthoyl)-3,3,3-trifluoroacetone (NTFA), and 4,4,4-Trifluoro-1-phenyl-1,3-butanedione (BTFA).

7. The phosphor material according to claim 1, wherein the antenna molecule is complexed to the lanthanide dopant, said complex having the formula Ln[ANT]n, where Ln is a member of the lanthanide series of elements, ANT is an antenna molecule and n is a whole number integer, optionally wherein n is from 3 to 300, such as 30, and optionally wherein Ln[ANT]ₙ is selected from one or more of the group consisting of Eu[TTA]₃, Sm[TTA]₃, Dy[TTA]₃, Tb[TTA]₃, Eu[BTFA]₃, Eu[NTFA]₃, optionally wherein Ln[ANT]ₙ is selected from one or more of the group consisting of Eu[TTA]₃, Sm[TTA]₃, and Tb[TTA]₃ (e.g. Eu[TTA]₃, and Tb[TTA]₃, such as and Eu[TTA]₃.

8. (canceled)

9. The phosphor material according to claim 1, to 8, wherein the molar ratio of the lanthanide dopant to the antenna molecule is from 1:300 to 1:3, such as from 1:200 to 1:10, such as from 1:100 to 1:20, such as about 1:30.

10. The phosphor material according to claim 1, wherein the phosphor material further comprises a co-dopant material, optionally wherein the co-dopant material is selected from one or more of the group consisting of Sr²⁺ and Ca²⁺.

11. The phosphor material according to claim 1, wherein the lanthanide dopant is present in an amount of from 20 to 200 nmol per mg dry spore biomass.

12. A composite material comprising:
a phosphor material as described in claim 1; and a bulk material, wherein the bulk material is selected from one or more of the group consisting of a resin, a coating, an adhesive, and an encapsulating material and the bulk material is optically transparent or optically translucent to the excitation and emission wavelengths of the phosphor material.

13. The composite material according to claim 12, wherein the bulk material comprises an adhesive, where the adhesive is one that is optically transparent to the excitation and emission wavelengths of the phosphor material, optionally wherein the adhesive is Norland™ 88.

14. A modified light emitting diode, comprising:
a light emitting diode, having a light emitting diode bulb section; and
a cured composite material comprising a phosphor material as described in claim 1, and an optically clear adhesive covering a surface of the light emitting diode section, such that light from the light emitting diode passes through the cured composite material.

15. The modified light emitting diode according to claim 14, wherein the light emitting diode is an ultraviolet light emitting diode.

16. A method of forming a phosphor material as described in claim 1, the method comprising the steps of:
(a) providing a plurality of bacterial spores of the Bacillus and Clostridium genera; and
(b) adding a lanthanide material to the plurality of bacterial spores to provide the phosphor material, wherein the lanthanide material is either a lanthanide salt or a lanthanide complexed to an antenna molecule.

17. The method according to claim 16, wherein the lanthanide material is provided in a solvent to the bacterial spores.

18. The method according to claim 17, wherein the solvent comprises water and ethanol, optionally wherein ethanol forms from 1 to 50% vol/vol, such as about 3% vol/vol.

19. The method according to claim 17, wherein the lanthanide material is provided in the solvent at a concentration of from 0.005 mM to 10 mM, such as from 0.01 mM to 3 mM, such as from 0.015 mM to 1.5 mM, such as from 0.15 mM to 0.75 mM.

20. The method according to claim 17, wherein the antenna molecule, when present, is provided in an amount of from 0.1 mM to 10 mM, such as about 4.5 mM.

21. The method according to claim 17, wherein the method further comprises the step of substantially removing the solvent from the phosphor material.
